(19) United States
(12) Patent Application Publication
(10) Pub. No.: US 2015/0057531 A1
(43) Pub. Date: Feb. 26, 2015

(54) SOFT TISSUE LESION EXCISION GUIDE

(71) Applicants: Tera Dennis, Roseville, CA (US); Jack Friedlander, Rocklin, CA (US)

(72) Inventors: Tera Dennis, Roseville, CA (US); Jack Friedlander, Rocklin, CA (US)

(21) Appl. No.: 14/469,130

(22) Filed: Aug. 26, 2014

Related U.S. Application Data
(60) Provisional application No. 61/869,825, filed on Aug. 26, 2013.

(51) Int. Cl. A61B 10/02; A61B 6/12

(52) U.S. Cl. CPC A61B 10/02; A61B 6/12
USPC 600/424; 600/564

(57) ABSTRACT

The present invention is a device, system and method for surgical soft tissue lesion excision. The device is designed to be placed into a soft tissue lesion preferably under radiologic guidance and deployed. Once deployed, the shape memory wires of the soft tissue encompassing element form a wire cage surrounding the lesion which acts as an excision guide, encompassing and protecting the lesion during surgical excision of the lesion.

This application claims the benefit of U.S. Provisional Application No. 61/869,825, filed Aug. 26, 2013, entitled SOFT TISSUE LESION EXCISION GUIDE, the disclosure of which is herein incorporated by reference.

### BACKGROUND OF THE INVENTION

1. Field of the Invention

The present invention relates generally to a device, system and method for performing surgical soft tissue lesion excision, more specifically, to a device, system and method for surgical soft tissue lesion excision performed after radiologic localization of the tissue lesion to be excised.

2. Description of the Related Art

In current practice, a patient with a radiologically identified lesion, for example, a breast lesion, which cannot be palpated by a physician, and thus cannot be surgically removed without some sort of guide or marker, is marked by placement of a radiologically guided wire which is placed into the tissue of the radiologically identified lesion. Subsequently, a surgeon during an operation, makes an incision and follows the guide wire down to the lesion, then excises the lesion. There are a number of inherent problems with this procedure as currently practiced. The first is that the currently available guide wires are easily dislodged from the lesion during manipulation during the operation. Second, the guide wire, as currently used, marks only the approximate center of the lesion. As the lesion is surrounded by tissue, and often not palpable by the surgeon's fingers, thus, the lesion can be inadvertently cut into, incompletely removed, or missed altogether. There is substantial need in the field for a radiologically guided marker which also surrounds and encompasses the lesion to be excised, preventing dislodging, cutting into the lesion, incompletely removing the lesion, or missing the lesion altogether.

### SUMMARY OF THE INVENTION

In preferred embodiments, the present invention comprises a radiologically placed soft tissue lesion excision device and system used to mark, encompass, and provide tactile feedback to the surgeon during the surgical removal of a soft tissue lesion.

In some embodiments, the device and system comprise a soft tissue encompassing element, an insertion element, a deployment element and a removal element. The soft tissue encompassing element comprises three or more shape memory wires, preferably formed from nitinol, or any other shape memory material. Each wire being formed such that it has a straight proximal section and a curved distal section, the curved distal section being pre bent in a generally curvilinear configuration and having the ability to be straightened fully, and then return to its original curvilinear configuration. The three or more shape memory wires are formed into a fixed arrangement such that the straight portions of the wires are generally parallel to and fixed to one another at a proximal end, forming a central axis, and the planes described by the curvilinear portions of the wires each being generally parallel to one another, perpendicular to the central axis, and spaced radially equidistant from one another.

For example, in a device comprising three wires, the curved portion of each wire would describe a vertical plane generally perpendicular to the central axis formed by the fixed straight portion of the wire. The planes described by the curved portions of the three wires would each be generally parallel to one another and spaced approximately one hundred and twenty degrees from one another. If four wires are used the planes described by the curved portions of the four wires would each be approximately ninety degrees from one another. The central straight portions of the wires being fixed to one another, or to a fixation element, such that the wires maintain their planar orientation rigidly. In a preferred embodiment, an extension wire is fixed to the proximal end of the three or more shape memory wires, or to the fixation element and is configured to extend outward through the skin of a patient and functional to act as a guide for the surgeon to locate the distal end of the encompassing element during an operation. In another embodiment, the extension wire is absent and the three or more shape memory wires are of a length that they can extend out from the lesion past the skin of a patient and act as a guide for the surgeon to locate the distal end of the encompassing element during an operation.

An insertion element comprises a hollow tube with an inner diameter large enough to admit the soft tissue encompassing element within said inner diameter, and sharp or beveled at one end to allow it to penetrate the tissue through which it is to be inserted. In another embodiment there is an infusion port connected to the inner channel of the hollow tube which may be used to administer a local anesthetic agent prior to deployment of the encompassing element. In another embodiment there are perforations in the distal end of the hollow tube functional to allow the infused liquid into the tissue lateral to the soft tissue lesion.

A deployment element comprises a second hollow tube, with an outer diameter small enough to fit inside the inner diameter of the insertion element and with an inner diameter both large enough to admit the extension wire of the encompassing element and small enough to prevent the three or more shape memory wires from passing through it. At a proximal end is a flattened portion functional to be pressed upon by a physician's finger.

A removal element comprises a hollow tube with an inner diameter large enough to admit the soft tissue encompassing element within said inner diameter, and with a conical bevel at one end configured to guide the curvilinear portions of the encompassing element into the inner diameter of the removal element allowing the encompassing element to be removed from the soft tissue lesion it has been inserted into following surgical excision of the lesion.

In a first stored configuration, the encompassing element is inserted into the inner diameter of the insertion element with the curvilinear portions of the shape memory wires having a generally straight configuration. The deployment element is inserted into the proximal end of the inner diameter of the insertion element such that the extension wire resides inside the inner diameter of the deployment element and the distal tip of the deployment element abuts the proximal ends of the three or more shape memory wires. In this configuration, the apparatus may be placed through the skin of a patient and into the approximate center of the soft tissue lesion, preferably via radiologic guidance. Once located into the approximate center of the lesion, the deployment element is pushed into the insertion element, deploying the encompassing element outward through the sharpened tip of the insertion element. As the encompassing element is deployed, the tips of the curvilinear portions of the shape memory wires penetrate the tissue and while traveling through the tissue, resume their curvilinear configuration surrounding and encompassing the soft tissue lesion. Once fully deployed, the insertion element and deployment element are withdrawn from the tissue, leaving the encompassing element in place around the tissue and the extension wire exiting from and extending outwardly past the surface of the skin. The patient would then be taken to an operating room and a surgeon would make an incision in the skin, then follow the extension wire down to the encompassing element surrounding the lesion. The surgeon would then excise the lesion encompassed within the encompassing element by cutting along the outer diameter formed by the shape memory wires in their curvilinear configuration surrounding the lesion. Once the lesion has been excised, the removal element is slid down the proximal portion of the encompassing element and the encompassing element pulled into the inner diameter of the removal element, removing the encompassing element from the soft tissue lesion allowing for pathological analysis of the lesion.

### BRIEF DESCRIPTION OF THE DRAWINGS

The present invention will be readily understood by the following detailed description in conjunction with the accompanying drawings, wherein like reference numerals designate like structural elements, and in which:

FIG. 1 is an exploded perspective view of an embodiment showing one example of a soft tissue lesion excision device according to the present invention;

FIG. 2 is a side view of an example of a single shape memory wire of the soft tissue lesion excision device in its deployed curvilinear conformation;

FIG. 3 is a perspective view of an example of a single shape memory wire of the soft tissue lesion excision device;

FIG. 4 is a side view of an example of a soft tissue encompassing element comprising three or more shape memory wires and an extension wire;

FIG. 5 is a perspective view of an example of the soft tissue encompassing element;

FIG. 6 is an end on view of an example of the soft tissue encompassing element;

FIG. 7 is a top perspective view of an example of the soft tissue encompassing element;

FIG. 8 is a perspective view of an example of an insertion element of the soft tissue biopsy marking and encompassing device;

FIG. 9 is a side view of an example of a deployment element of the soft tissue biopsy marking and encompassing device;

FIG. 10 is a perspective view of an example of the deployment element;

FIG. 11 is a perspective view of an example of the removal element;

FIG. 12 is a cutaway view of the distal end of an example of the removal element;

FIG. 13A is a side view of an example of a soft tissue biopsy marking and encompassing device in its first loaded position;

FIG. 13B is a side view of an example of a soft tissue biopsy marking and encompassing device in its second partially deployed position; and

FIG. 13C is a side view of an example of a soft tissue biopsy marking and encompassing device in its third fully deployed position.

### DETAILED DESCRIPTION OF THE INVENTION

The following description is provided to enable any person skilled in the art to make and use the invention and sets forth the best modes contemplated by the inventor for carrying out the invention. Various modifications, however, will remain readily apparent to those skilled in the art. Any and all such modifications, equivalents and alternatives are intended to fall within the spirit and scope of the present invention. In describing the orientation of the device, the term proximal will refer to that end of the described part closest to the portion.

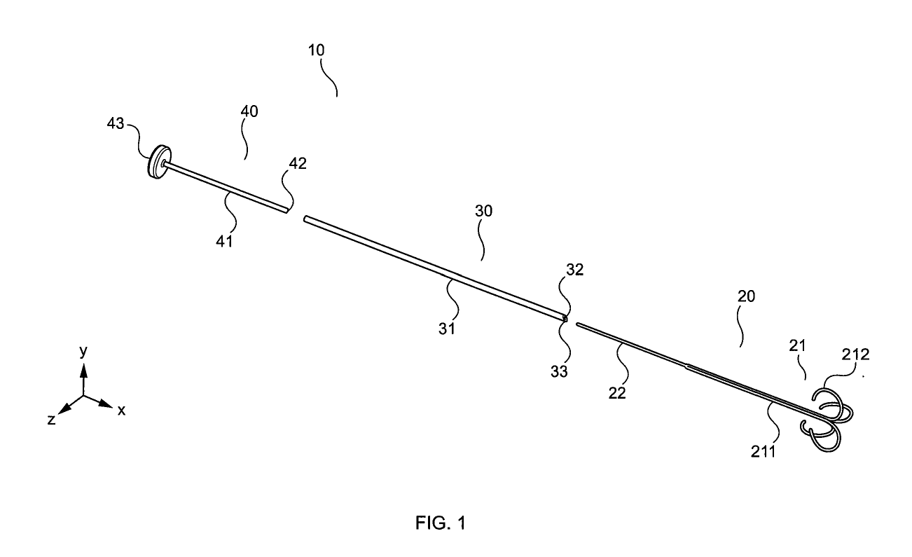

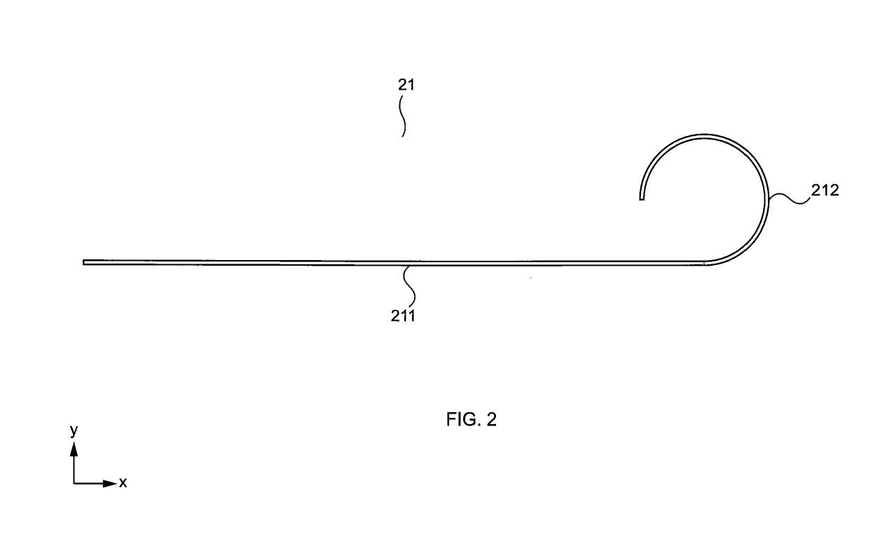

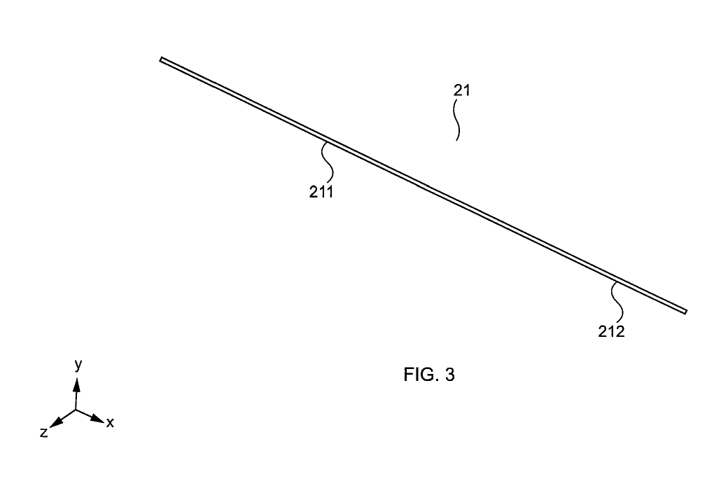

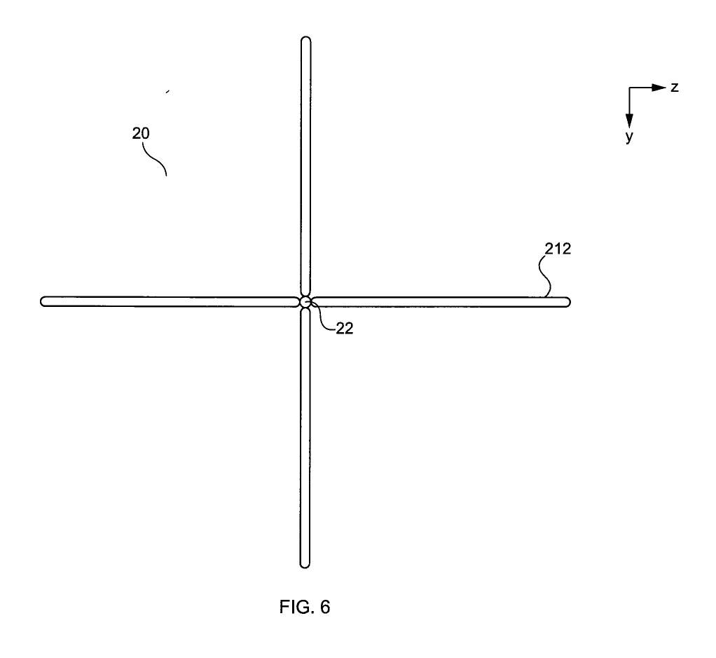

FIG. 1 is an exploded perspective view of a soft tissue mass excision guide system 10 including an encompassing device (sometimes referred to as a “soft tissue biopsy marking and encompassing device” or the “apparatus” or the “device”) according to an embodiment of the present invention. In preferred embodiments, the apparatus 10 includes a soft tissue marking and encompassing element (encompassing element) 20 comprising three or more shape memory wires 21 constructed preferably of nitinol, or other similar shape memory material, including elastic or super elastic shape memory material. The term “wire” herein refers to the shape memory material used to form the encompassing elements, regardless of whether they are formed of metal wires, or of non-metal material, such as an elastic polymer. The shape memory wires 21 each having a proximal straight portion 211 and a distal curvilinear portion 212 (detailed in FIG. 2), the curvilinear portion functional to transition between a first straight stored configuration depicted in FIG. 3 and a second curvilinear deployed configuration depicted in FIG. 2. The proximal straight portions 211 of the three or more shape memory wires 21 being aligned in parallel with one another along a central axis, arranged in a radial pattern around said central axis and fixed to one another such that the curvilinear portions 212 are oriented substantially perpendicular to said central axis and generally equidistant from one another as depicted in FIG. 6, functional to encompass a soft tissue mass and provide a dissection guide for a surgeon surgically removing said soft tissue mass from the body of a patient. Fixed to the proximal end of the three or more shape memory wires 21 is an extension wire 22 functional to extend outward through the skin of the patient after placement and to provide a directional guide for a surgeon to accurately locate the soft tissue mass encompassed by the soft tissue marking and encompassing element 20. Fixation of the three or more shape memory wires 21 to one another and to the extension wire 22 can be accomplished by various techniques, including but not limited to welding, soldering, encasement in an epoxy resin, or molded to one another using plastic or metal molding. The three or more shape memory wires 21 may have any of a number of cross sectional shapes including round, squared, triangular or pie shaped such that the three or more shape memory wires are rigidly fixed in their planar orientation in the formation of the soft tissue encompassing element 20.

An insertion element 30 comprises a hollow tube having an outer wall 31 and an inner channel 32. The insertion element is preferably sharpened at one end 33 with the diameter of the inner channel 32 being large enough to admit the full diameter of the soft tissue encompassing element 20.

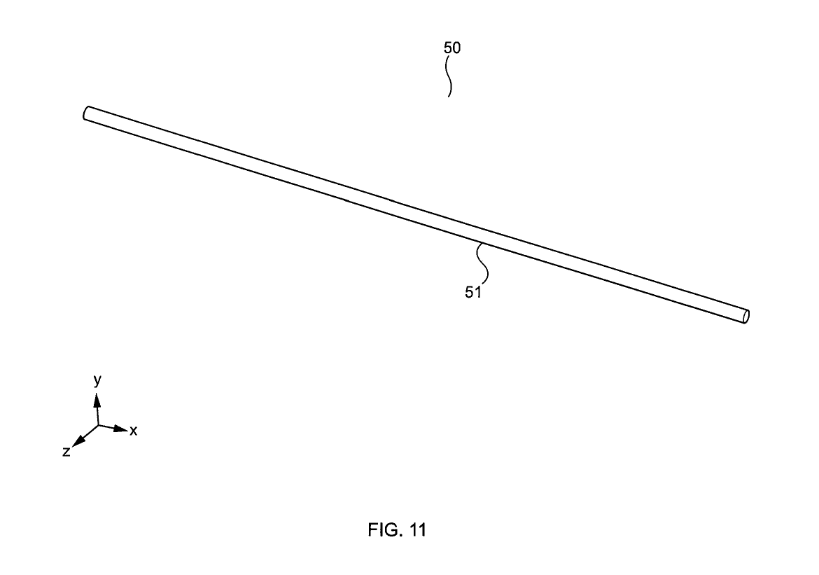

As illustrated in FIG. 11, a removal element 50 comprises a hollow tube having an outer wall 51 and an inner channel 52 with the diameter of the inner channel 52 being large enough to admit the full diameter of the soft tissue encompassing element 20. The inner channel is preferably flared at a distal end 53 forming a conical shape functional to guide the distal curvilinear portions of the shape memory wires 212 into the inner channel 52 of the removal element.

In preferred embodiments, a deployment element 40 comprises a second hollow tube, having an outer wall 41 and an inner channel 42 and a widened plunger 43 on its proximal end. The deployment element being formed such that the diameter of the outer wall 41 is smaller than the inner diameter of the channel 32 of the insertion element 30 and functional to slide easily inside the inner channel 32 of the insertion element 30. The inner channel of the deployment element 42 is formed such that it is larger than the diameter of the extension wire 22 but smaller than the diameter of the shelf 23 formed by the combination of the proximal ends of the three or more shape memory wires 21 allowing the distal end of the deployment element to abut said shelf 23 of the shape memory wires 22 and functional to slide the marking and encompassing element distally into the tissue when pressure is applied to the plunger 43.

FIG. 2 is a side view of an example of a shape memory wire 21 in its second curvilinear deployed configuration detailing the proximal straight portion 211 and a distal curvilinear portion 212.

FIG. 3 is a perspective view of an example of a shape memory wire 21 showing the distal curvilinear portion in its first straight configuration.

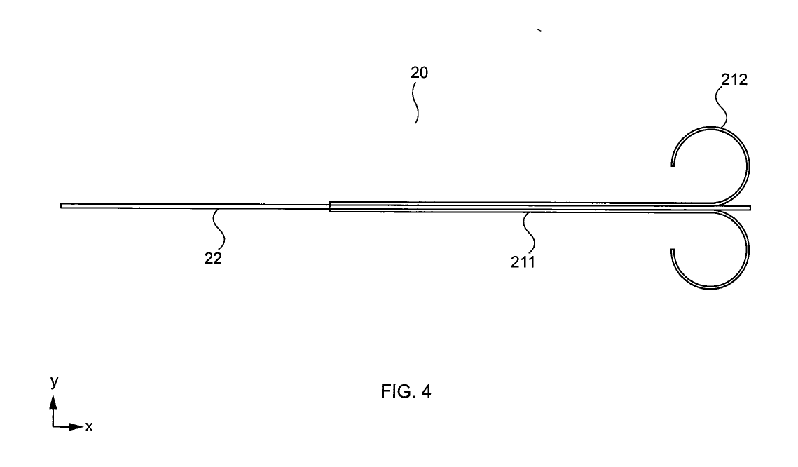

FIG. 4 is a side view of an example of a soft tissue encompassing element 20 in its deployed configuration, the three or more shape memory wires 21 in their second curvilinear configuration, the extension wire 22 extending from the proximal end of the three or more fixed shape memory wires 21.

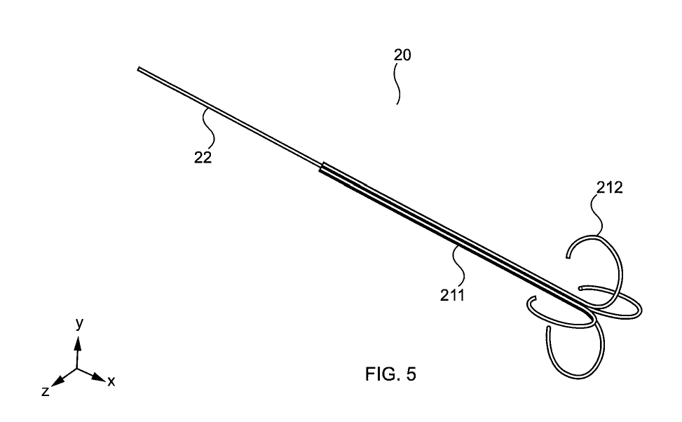

FIG. 5 is a perspective view of an example of a soft tissue encompassing element 20 in its deployed configuration, the proximal straight portions 211 of the three or more shape memory wires 21 being aligned in parallel with one another along an axis, arranged in a radial pattern around said axis and fixed to one another or optionally to an extension wire 22.

FIG. 6 is a distal end on view of an example of a soft tissue encompassing element 20 showing the curvilinear portions 212 the shape memory wires 21 oriented substantially perpendicular to the central axis, generally equidistant from one another and functional to encompass a soft tissue mass and provide a dissection guide for a surgeon surgically removing said soft tissue mass.

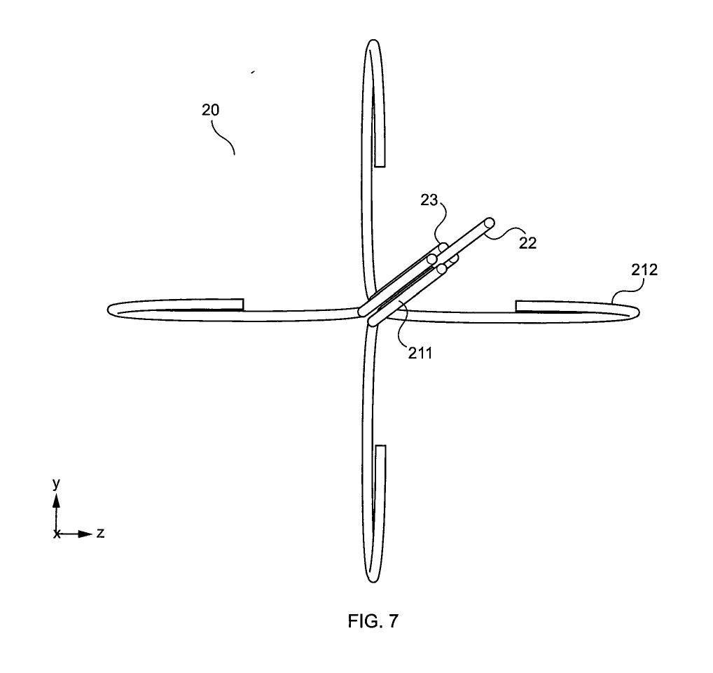

FIG. 7 is a perspective proximal end on view of an example of a soft tissue encompassing element 20 detailing the shelf 23 formed by the proximal ends of the three or more shape memory wires 21 against which the distal end of the deployment element 40 abuts in order for the deployment element 40 to push the soft tissue encompassing element 20 out of the distal end of the insertion element 30 during deployment of the soft tissue encompassing element 20. Fixation of the three or more shape memory wires 21 to one another and to the extension wire being accomplished by any number of various techniques, including but not limited to welding, soldering, encasement in an epoxy resin or molded to one another using plastic or metal molding.

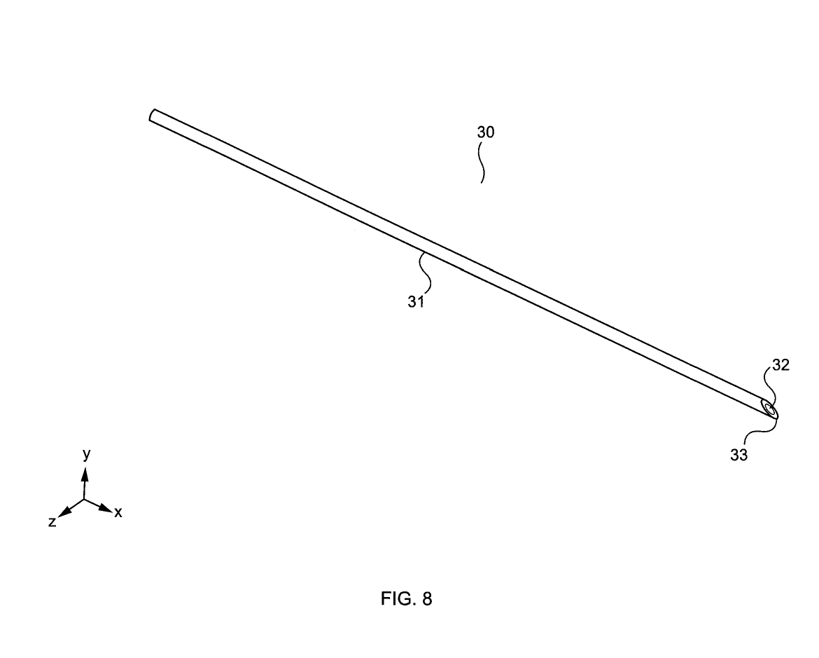

FIG. 8 is a perspective view of an example of the insertion element 30 as shown by example in FIG. 1 detailing the outer wall 31, the inner channel 32, and the sharpened distal end 33.

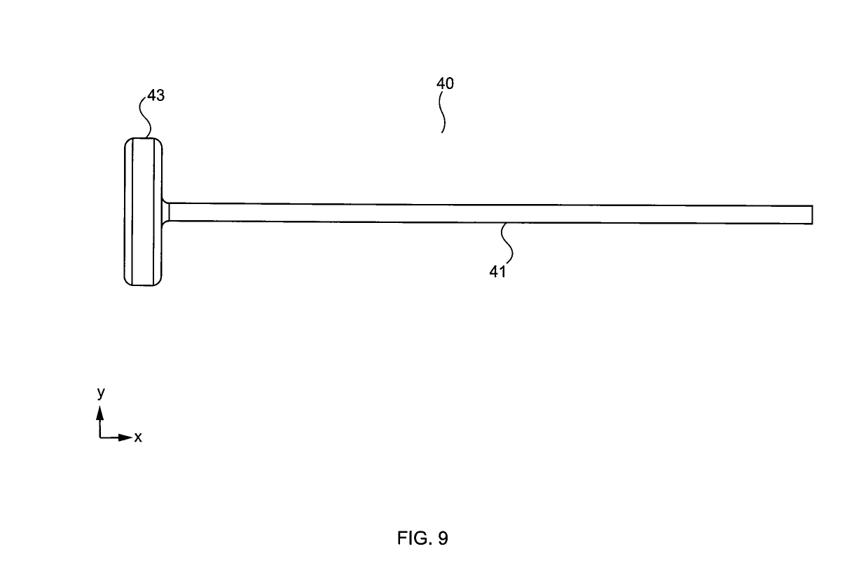

FIG. 9 is a side view of an example of a deployment element 40 showing the outer wall 41 and plunger 43.

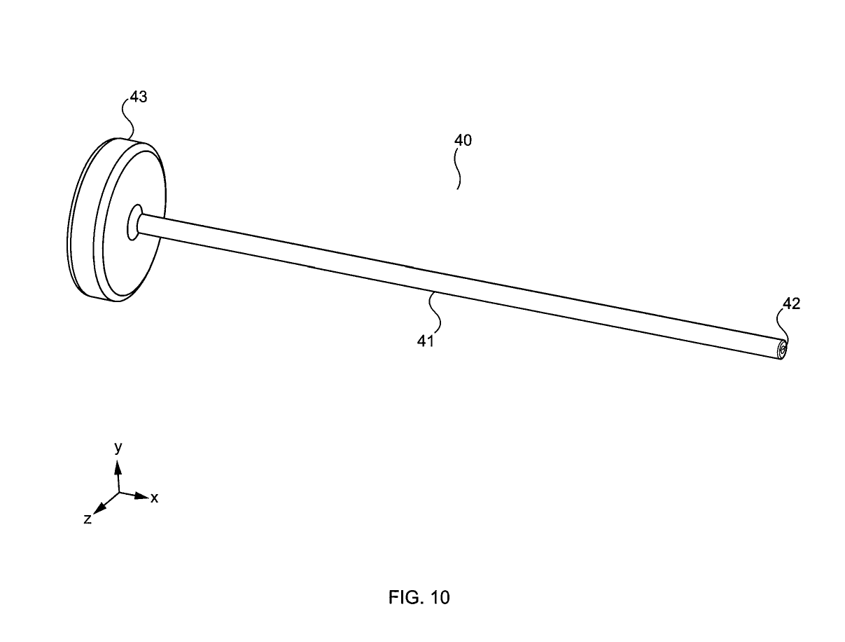

FIG. 10 is a perspective view of an example of a deployment element 40 detailing the outer wall 41, inner channel 42 and plunger 43.

FIG. 11 is a perspective view of an example of a removal element 50.

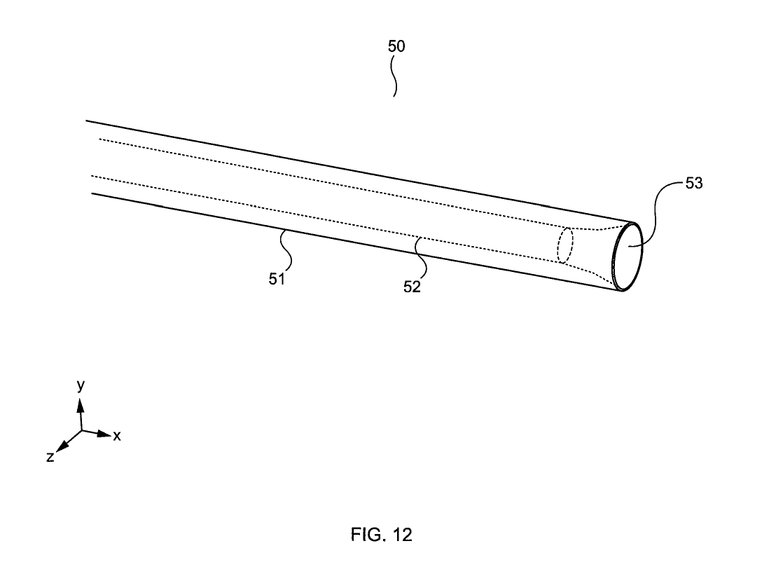

FIG. 12 is a cutaway view of an example of a removal element 50 detailing the conical shape at the distal end 53 of the inner channel 52.

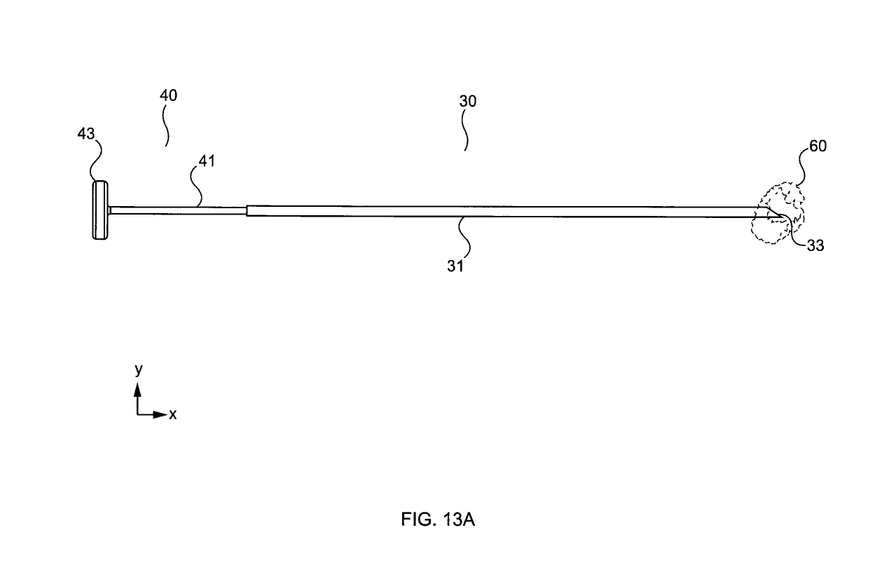

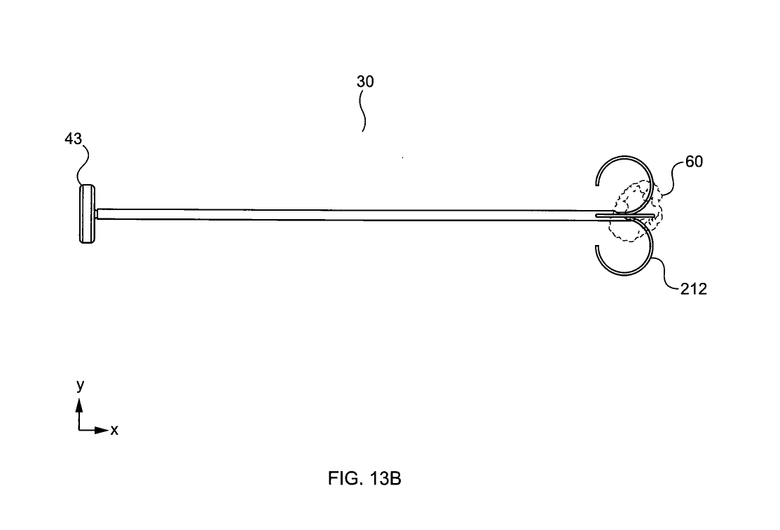

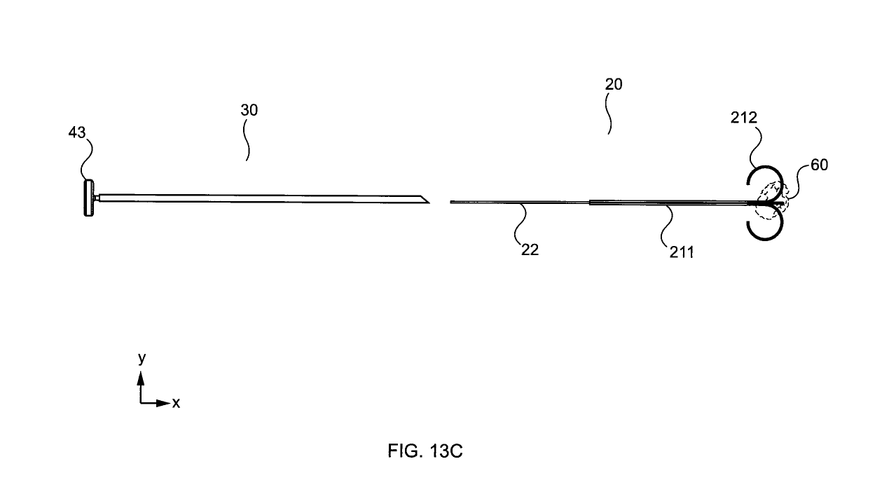

FIGS. 13 A-C illustrate one working example of the operation of the soft tissue biopsy marking and encompassing device 10. FIG. 13A shows the soft tissue biopsy marking and encompassing device 10 is in a first stored configuration with the curvilinear portions 212 of the shape memory wires 21 in their first straight configuration and with the marking and encompassing element 20 inserted into the inner channel of the insertion element 30. The deployment element 40 having been inserted through the opposite end of the insertion element 30 and having the extension wire 22 contained within its inner channel 32 such that the distal end of the deployment element 40 abuts the proximal end 23 of the three or more shape memory wires 21. In this configuration, the apparatus 10 is inserted, preferably under radiologic guidance, through the overlying skin and soft tissue of a patient, and into the soft tissue lesion 60 to be excised. Once it is radiologically confirmed that the tip of the insertion element 30 lies at or near the center of the soft tissue lesion 60, the plunger 43 is depressed moving the deployment element 40 distally against the ends of the shape memory wires at their shelf 23 and pushing the soft tissue encompassing element 20 outward through the sharpened tip of the insertion element 33. As the encompassing element 20 is deployed, the tips of the curvilinear portions of the shape memory wires 212 penetrate the tissue and while traveling through the tissue, resume their second curvilinear configuration surrounding and encompassing the soft tissue lesion 60.

Once fully deployed, the insertion element 30 and deployment element 40 are withdrawn from the tissue, leaving the encompassing element 20 in place around the soft tissue lesion 60 and the extension wire 22 extending outwardly past the surface of and exiting from the skin of the patient. The patient would then be taken to an operating room and a surgeon would make an incision in the skin, then follow the extension wire 22 down to the encompassing element 20 surrounding the lesion 50. The surgeon would then be able to safely excise the lesion encompassed within the encompassing element by cutting around the outer diameter formed by the shape memory wires 21 in their curvilinear configuration surrounding the lesion 60. Following excision of the soft tissue lesion 60 the encompassing element 20 may be removed from the soft tissue lesion 60 by sliding the removal element 50 down over first the extension wire 22 then over the proximal straight portion of the encompassing element 211. The encompassing element 20 is then drawn up into first the flared conical end 53 of the removal element 50 and then into the inner channel of the removal element 52, the flared conical end functional to guide the curvilinear portions of the shape memory wires 212 into a more straightened configuration, facilitating the entry of the curvilinear portions of the shape memory wires 212 into the inner channel 52.

Those skilled in the art will appreciate that various adaptations and modifications of the just described preferred embodiments can be configured without departing from the scope and spirit of the invention. Therefore, it is to be understood that, within the scope of the appended claims, the invention may be practiced other than as specifically described herein.

What is claimed is:

1. A soft tissue biopsy marking and encompassing device comprising:
an encompassing element comprising at least three shape memory wires, each wire having a straight proximal section and a curved distal section;
wherein each curved distal section is formed in a generally curvilinear shape and can be straightend, and return to the original curviliner shape.

2. The soft tissue biopsy marking and encompassing device of claim 1, wherein the at least three shape memory wires are formed in a fixed arrangement such that the straight sections of the wires are generally parallel and fixed to one another at a proximal end forming a central axis.

3. The soft tissue biopsy marking and encompassing device of claim 2, wherein planes defined by the curved sections are perpendicular to the central axis and are spaced radially equidistant from each other.

4. The soft tissue biopsy marking and encompassing device of claim 3, wherein the at least three shape memory wires comprise three wires spaced 120 degrees from each other.

5. The soft tissue biopsy marking and encompassing device of claim 3, wherein the at least three shape memory wires comprise four wires spaced 90 degrees from each other.

6. The soft tissue biopsy marking and encompassing device of claim 3, wherein the at least three shape memory wires are of sufficient length such that they can extend out from a lesion past the skin of a patient to act as a guide.

7. The soft tissue biopsy marking and encompassing device of claim 3, wherein the shape memory wires comprise nitinol.

8. The soft tissue biopsy marking and encompassing device of claim 3, further comprising an extension wire fixed to the proximal end of the at least three shape memory wires, the extension wire is of sufficient length to extend out past the skin of a patient to act as a guide.

9. The soft tissue biopsy marking and encompassing device of claim 8, further comprising an insertion element, the insertion element comprising a first hollow tube having an inner diameter large enough to admit the at least three shape memory wires.

10. The soft tissue biopsy marking and encompassing device of claim 9, wherein the first hollow tube has a sharp end to penetrate tissue.

11. The soft tissue biopsy marking and encompassing device of claim 9, wherein the first hollow tube has a beveled end to penetrate tissue.

12. The soft tissue biopsy marking and encompassing device of claim 9, wherein the first hollow tube further comprises an infusion port, and perforations at a distal end of the first hollow tube.

13. The soft tissue biopsy marking and encompassing device of claim 9, further comprising a deployment element, the deployment element comprising a second hollow tube, having an outer diameter small enough to fit inside the inner diameter of the insertion element, and having an inner diameter large enough to admit the extension wire, and small enough to prevent the at least three shape memory wires from passing through the deployment element.

14. The soft tissue biopsy marking and encompassing device of claim 13, further comprising a removal element, the removal element comprising a third hollow tube having an inner diameter large enough to admit the encompassing element within the inner diameter, and with a conical bevel at one end configured to guide the curvilinear portions of the encompassing element into the inner diameter of the removal element allowing the encompassing element to be removed from the soft tissue lesion it has been inserted into following surgical excision of the lesion.

15. A soft tissue biopsy marking and encompassing system comprising:
an encompassing element comprising at least three shape memory wires, each wire having a straight proximal section and a curved distal section;
an insertion element, the insertion element comprising a first hollow tube having an inner diameter large enough to admit the at least three shape memory wires;
a deployment element, the deployment element comprising a second hollow tube, having an outer diameter small enough to fit inside the inner diameter of the insertion element; and
a removal element, the removal element comprising a third hollow tube having an inner diameter large enough to admit the encompassing element within the inner diameter, and with a conical bevel at one end.

16. The soft tissue biopsy marking and encompassing system of claim 15, wherein the at least three shape memory wires are formed in a fixed arrangement such that the straight sections of the wires are generally parallel and fixed to one another at a proximal end forming a central axis.

17. The soft tissue biopsy marking and encompassing system of claim 16, wherein planes defined by the curved sections are perpendicular to the central axis and are spaced radially equidistant from each other.

18. The soft tissue biopsy marking and encompassing system of claim 17, wherein the at least three shape memory wires comprise three wires spaced 120 degrees from each other.

19. The soft tissue biopsy marking and encompassing system of claim 17, wherein the at least three shape memory wires comprise four wires spaced 90 degrees from each other.

20. The soft tissue biopsy marking and encompassing system of claim 17, wherein the at least three shape memory wires are of sufficient length such that they can extend out from a lesion past the skin of a patient to act as a guide.

21. The soft tissue biopsy marking and encompassing system of claim 15, wherein the shape memory wires comprise nitinol.

22. The soft tissue biopsy marking and encompassing system of claim 17, further comprising an extension wire fixed to the proximal end of the at least three shape memory wires, the extension wire is of sufficient length to extend out past the skin of a patient to act as a guide.

23. The soft tissue biopsy marking and encompassing system of claim 17, wherein the first hollow tube has a sharp end to penetrate tissue.

24. The soft tissue biopsy marking and encompassing system of claim 17, wherein the first hollow tube has a beveled end to penetrate tissue.

25. The soft tissue biopsy marking and encompassing system of claim 17, wherein the first hollow tube further comprises an infusion port, and perforations at a distal end of the first hollow tube.

26. The soft tissue biopsy marking and encompassing system of claim 22, wherein the deployment element comprises an outer diameter small enough to fit inside the inner diameter of the insertion element, and having an inner diameter large enough to admit the extension wire, and small enough to prevent the at least three shape memory wires from passing through the deployment element.

27. A method for soft tissue biopsy marking and encompassing comprising:
inserting an encompassing element, the encompassing element having at least three shape memory wires, each wire comprising a curved section and a straight section, into an insertion element;
inserting a deployment element into a proximal end of the insertion element, such that a distal end of the deployment element abuts proximal ends of the at least three shape memory wires;
inserting the deployment element through a patient's skin into an approximate center of a lesion;
pushing the deployment element into the insertion element to deploy the encompassing element;
withdrawing the insertion element and deployment element.

28. The method of claim 27, wherein the step of inserting an encompassing device comprises inserting the deployment element through a patient's skin comprises inserting the deployment element under radiologic guidance.
